(19) United States
(12) Patent Application Publication
(10) Pub. No.: US 2024/0345107 A1
(43) Pub. Date: Oct. 17, 2024

(54) METHOD FOR QUALITATIVE AND/OR QUANTITATIVE DETECTION OF SUBSTANCES CONTAINED IN A HEMP PLANT AND KIT FOR USE THEREIN

(71) Applicant: Luxcan Innovation S.A., Luxemburg (LU)

(72) Inventor: Fritz SCHMITT, Steinheim (LU)

(21) Appl. No.: 18/751,821

(22) Filed: Jun. 24, 2024

Related U.S. Application Data
(62) Division of application No. 17/421,046, filed on Jul. 7, 2021.

(30) Foreign Application Priority Data
Jan. 7, 2019 (DE) 10 2019 000 016.1
Jan. 7, 2019 (DE) 10 2019 000 018.8
Jan. 15, 2019 (DE) 10 2019 000 199.0

(51) Int. Cl. G01N 33/94; A24F 7/04; A24F 40/30; A24F 40/42; A24F 40/465; A24F 40/51; A24F 40/57; A24F 42/10; A24F 47/00; A61J 3/10; A61K 36/185; A61M 11/02; A61M 11/04; A61M 15/00; B01L 3/00; B30B 11/04; B30B 15/30; G01N 33/52

(52) U.S. Cl. CPC G01N 33/948; A24F 7/04; A24F 40/30; A24F 40/42; A24F 40/465; A24F 40/51; A24F 40/57; A24F 42/10; A24F 47/00; A61J 3/10; A61K 36/185; A61M 11/02; A61M 11/047; A61M 15/0021; A61M 15/0028; B01L 3/502; B01L 3/52; B01L 3/523; B30B 11/04; B30B 15/302; G01N 33/52; A61M 11/041; A61M 2205/3368; A61M 2205/3686; A61M 2205/6054; A61M 2205/6072; A61M 2205/609; A61M 2205/75; A61M 2206/20; B01L 2300/069; B01L 2300/0835; B01L 2300/168; B01L 2300/1816

(57) ABSTRACT

A method for qualitatively and/or quantitatively detecting one or more substances contained in the hemp plant using a kit having a) an ampoule; b) a material comprising a hemp plant or parts thereof; and c) a color indicator capable of reacting by contacting the hemp plant and/or at least a part thereof to change the color of the color indicator, wherein the material and the color indicator are disposed in the ampoule.

### CROSS-REFERENCE TO RELATED APPLICATIONS

This application is a divisional patent application of U.S. Ser. No. 17/421,046 filed Jul. 7, 2021 which is a U.S. National Phase Application under 35 U.S.C. 371 of International Application No. PCT/DE2019/101064, filed on Dec. 10, 2019, which claims the benefit of German Application No. 10 2019 000 0199.0, filed on Jan. 15, 2019, and German Application No. 10 2019 000 016.1, filed on Jan. 7, 2019, and German Application No. 10 2019 000 018.8, filed on Jan. 7, 2019. The entire disclosures of the above applications are incorporated herein by reference.

### BACKGROUND

This section provides background information related to the present disclosure which is not necessarily prior art.

### Technical Field

The present invention relates to a method for the qualitative and/or quantitative detection of substances contained in the hemp plant. The invention further relates to a kit for use in this method.

### Discussion

The legalization of cannabis and its use as a medicine raise many questions of quality assurance by the producer and distributor, especially pharmacists and physicians. In particular, it must be guaranteed by the pharmacist and verifiably documented according to pharmaceutical rules what the active ingredient content of a product placed on the market is.

Information on the content of THC and CBD is particularly problematic. As far as the content of these key substances is concerned, no fixed upper and lower limits are specified, but they should be known or should be able to be determined. The content of a batch should be within a range of +/−10 percent of the content declared on the package. This is due to the fact that the individual varieties sometimes differ considerably in terms of their content of tetrahydrocannabinol (THC) and cannabidiol (CBD). The fact that there can also be considerable differences within a variety from harvest to harvest is not taken into account. From a pharmaceutical point of view, this is unsatisfactory, especially in view of the fact that modified and strict conditions are to be expected in the future.

In EP 1 32 313 A2 cannabinoid detection methods for drug test kits based on diazonium reagents are described. The reaction is carried out on a filter paper and the reagents are applied in liquid form or as a spray. These and similar procedures are relatively cumbersome to perform. In addition, preparation of the sample material by means of measures such as drying, extraction, filtration and evaporation, is necessary in order to be able to carry out the corresponding determinations. This requires a considerable amount of work and time and can sometimes also lead to a change in the material composition of the sample material. In addition, these methods often require the use of toxic or highly corrosive chemicals, which is problematic for widespread use by the layperson.

### SUMMARY

This section provides a general summary of the disclosure, and is not a comprehensive disclosure of its full scope or all of its features.

Accordingly, it is one aspect of the present invention to provide a method for the qualitative and/or quantitative detection of substances contained in hemp plants which overcomes disadvantages of the prior art, in particular to provide a method which permits the simple and inexpensive analysis of substances which may be contained in hemp plants or other hemp-based products, in particular by persons who are inexperienced in chemical analysis, such as skilled personnel, pharmacists, physicians, clinical personnel, etc. In particular, the method according to the invention is intended to allow persons inexperienced in analysis, such as pharmacists, to determine the active ingredient content in hemp-based products with an accuracy that meets current requirements.

This task is solved by a kit comprising: a) an ampoule; b) a material comprising a hemp plant or parts thereof; and c) a color indicator capable of reacting by contacting the hemp plant and/or at least a part thereof to change the color of the color indicator, wherein the material and the color indicator are arranged in the ampoule.

The method according to the invention allows a simple qualitative and quantitative quality testing of hemp-or cannabis-based materials prior to marketing. This testing can be performed in a simple and safe manner even by the untrained person, such as a pharmacist, who can then ensure and certify the quality of the product he or she distributes.

The material comprises a hemp plant or a part thereof. A part of the hemp plant may also be understood as a single chemical compound isolated from the hemp plant, preferably a pharmaceutically active compound.

Cannabis (hemp), together with the genus Humulus (hops), belongs to the family Cannabisaceae, although Humulus does not contain cannabinoids. Within the genus Cannabis, a botanical and chemotaxonomic differentiation is made into the species Cannabis sativa Linnaeus, Cannabis indica LAM and Cannabis ruderalis or into the “collective species” Cannabis sativa L., consisting of the subspecies Cannabis sativa ssp. sativa and ssp. indica. In addition, cannabis is divided into a drug and fiber hemp, with the distinction based on the quantitative ratio of the main cannabinoids cannabidiol (CBD) and Δ9-tetrahydrocannabinol (Δ9-THC) (INN: dronabinol). Fiber hemp (also: commercial hemp, industrial hemp) is mainly used for industrial fiber production and may have a maximum Δ9-THC content of 0.2% (e.g. Germany et al.), while the drug type may have a Δ9-THC content of about 5-15% (marijuana, hashish). Cannabis sativa L. contains over 400 different constituents, of which more than 60 compounds belong to the class of cannabinoids. The most important cannabinoids are shown below:

Cannabigerol-like (CBG): Cannabigerol ((E)-CBG-C5), Cannabigerol monomethyl ether ((E)-CBGM-C5 A), Cannabinerolic acid A ((Z)-CBGA-C5 A), Cannabigerovarin ((E)-CBGV-C3), Cannabigerolic acid A ((E)-CBGA-C5 A), Cannabigerolic acid A monomethyl ether ((E)-CBGAM-C5 A), Cannabigerovaric acid A ((E)-CBGVA-C3 A);

Cannabichromene-like (CBC): cannabichromene (CBC-C5), cannabichromic acid A (CBCA-C5 A), cannabichromevarin (CBCV-C3), cannabichromevarinic acid A (CBCVA-C3 A);

Cannabidiol-like (CBD): cannabidiol (CBD-C5), cannabidiol monomethyl ether (CBDM-C5), cannabidiol-C4 (CBD-C4), cannabidivarin (CBDV-C3), cannabidiorcol (CBD-C1), cannabidiolic acid (CBDA-C5), cannabidivaric acid (CBDVA-C3);

Cannabinodiol-like (CBND): Cannabinodiol (CBND-C5), Cannabinodivarin (CBND-C3);

Tetrahydrocannabinol-like (THC): Δ9-tetrahydrocannabinol (Δ9-THC-C5), Δ9-tetrahydrocannabinol-C4 (Δ9-THC-C4), Δ9-tetrahydrocannabivarin (Δ9-THCV-C3), Δ9-tetrahydrocannabiorcol (Δ9-THCO-C1), Δ9-tetrahydrocannabinolic acid (Δ9-THCA-C5 A), Δ9-tetrahydrocannabinolic acid B (Δ9-THCA-C5 B), Δ9-tetrahydrocannabinolic acid-C4 (Δ9-THCA-C4 A and/or B), Δ9-tetrahydrocannabivaric acid A (Δ9-THCVA-C3 A), Δ9-tetrahydrocannabiorcolic acid (Δ9-THCOA-C1 A and/or B), (−)-Δ8-trans-(6aR, 10aR)-Δ8-tetrahydrocannabinol (Δ8-THC-C5), (−)-Δ8-trans-(6aR,10aR)-tetrahydrocannabinolic acid A (Δ8-THCA-C5 A); (−)-(6aS, 10aR)-Δ9-tetrahydrocannabinol ((−)-cis-Δ9-THC-C5);

Cannabinol-like (CBN): cannabinol CBN-C5, cannabinol-C4 (CBN-C4), cannabivarin (CBN-C3), cannabinol-C2 (CBN-C2), cannabiorcol (CBN-C1), cannabinolic acid A (CBNA-C5 A), cannabinol methyl ether (CBNM-C5).

Cannabitriol-like (CBT): (−)-(9R,10R)-trans-cannabitriol ((−)-trans-CBT-C5), (+)-(9S,10S)-cannabitriol ((+)-trans-CBT-C5), (±)-(9R,10S/9S,10R)-cannabitriol ((±)-cis-CBT-C5), (−)-(9R,10R)-trans[10-0-Ethyl-cannabitriol] ((−)_{-trans-CBT-OEt-C5}), (±)-(9R,10R/9S,10S)-Cannabitriol-C3 ((±)_{-trans-CBT-C3}),8,9-dihydroxy-Δ6a(10a) tetrahydrocannabinol (8,9-di-OH-CBT-C5), cannabidiolic acid A (CBDA-C5 9-OH-CBT-C5 ester), (−)-(6aR,9S,10S,10aR)-9, 10-dihydroxy-hexahydrocannabinol, Cannabiripsol Cannabiripsol-C5, (−)-6a,7,10a-trihydroxy-Δ9-tetrahydrocannabinol ((−)-cannabitetrol), 10-Oxo-Δ6a(10a) tetrahydrocannabinol (OTHC);

Cannabielsoin-like (CBE): (5aS,6S,9R,9aR)-C5-cannabielsoine (CBE-C5), (5aS,6S,9R,9aR)-C3-cannabielsoine (CBE-C3), (5aS,6S,9R,9aR)-cannabielsoic acid A (CBEA-C5 A), (5aS,6S,9R,9aR)-cannabielsoic acid B (CBEA-C5 B), (5aS,6S,9R,9aR)-C3-cannabielsoic acid B (CBEA-C3 B), cannabiglendol-C3 (OH-iso-HHCV-C3), dehydrocannabifuran (DCBF-C5), cannabifuran (CBF-C5);

Isocannabinoids: (−)-Δ7-trans-(1R,3R,6R)-isotetrahydrocannabinol, (±)-Δ7-1,2-cis-(1R,3R,6S/1S,3S,6R)-isotetrahydrocannabivarin, (−)-Δ7-trans-(1R,3R,6R)-isotetrahydrocannabivarin;

Cannabicyclol-like (CBL): (±)-(1aS,3aR,8bR,8cR-cannabicyclol (CBL-C5), (±)-(1aS,3aR,8bR,8cR-cannabicyclic acid A (CBLA-C5 A), (±)-(1aS,3aR,8bR,8cR-cannabicyclovarin (CBLV-C3);

Cannabicitran-like (CBT): Cannabicitran (CBT-C5);

Cannabichromanone-like (CBCN): cannabichromanone (CBCN-C5), cannabichromanone-C3 (CBCN-C3), cannabicoumaronone (CBCON-C5).

In addition to the cannabinoids mentioned above, their associated carboxylic acids are found in the crude drug. These carboxylic acids are biosynthetic precursors.

Cannabis preparations exert a variety of therapeutic effects, including antispasmodic, analgesic, antiemetic, neuroprotective, anti-inflammatory, and effects in psychiatric disorders (Grotenhermen F, Müller-Vahl K: The therapeutic potential of cannabis and cannabinoids.

In Germany, a cannabis extract containing THC (dronabinol) and CBD in a 1:1 ratio (Nabiximols) has been approved for the treatment of moderate to severe therapy-resistant spasticity in multiple sclerosis (MS) as a sublingual spray (Sativex) since 2011.

Cannabidiol (CBD, CBD-C5) is the major non-psychotropic cannabinoid of the genus Cannabis and CBD is not a cannabinoid receptor agonist.

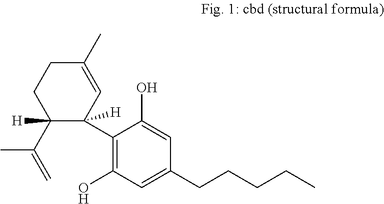 [SMILES: C=C(C)[C@@H]1CCC(C)=C[C@H]1c1c(O)cc(CCCCC)cc1O]

CBD can be produced synthetically (Michoulam R, Shvo Y., Hashish. I. The structure of cannabidiol, Tetrahedron. 1963, 19 (12), 2073).

In one embodiment, it may be provided that the material comprises a cannabis flower, marijuana, hashish, hashish oil, at least one cannabinoid, or a mixture thereof.

In various embodiments, the kit according to the invention (as well as the method according to the invention described below) can be used for the identification and quality determination of cannabis preparations such as marijuana, hashish, hashish oil and other cannabinoid-containing materials, for the determination of the degree of maturity of hemp plants, as well as for the differentiation of drug and also in the case of young hemp plants in fresh or also dried form.

In particular, it may be envisaged that the kit/process according to the invention is used for the chemical detection of various phenolic-like compounds, essential oils, resinoids, fresh plants, plant drugs, plant extracts and extracts and odorants, as well as for their identification and characterization and quality determination.

In a further embodiment, it may be provided that the color indicator comprises a color-forming substance preferably selected from the group consisting of true black, true blue salt, dibromoquinone chlorimide, dichloroquinone chlorimide, vanillin, salicylaldehyde, formaldehyde, acetaldehyde, p-dimethylaminobenzaldehyde, diethylaminobenzaldehyde, ferric chloride, aminophenol, aminoantipyrine, potassium ferricyanide, and a mixture of two or more thereof.

Likewise, it may be provided that the color indicator comprises at least one solvent preferably selected from the group consisting of water and monohydric or polyhydric alcohols.

In particular, it is possible to use one or more polyhydric alcohols alone or mixtures thereof as solvents, which on the one hand provide optimum extraction of the active ingredients from the material and on the other hand also good dissolving properties for the color-forming substance used and possibly other components of the color indicator and guarantee a trouble-free color reaction.

In a further embodiment, it may be provided that the color indicator further comprises a reagent that assists in reacting the material with the color-forming substance, wherein the reagent is preferably a basic compound, more preferably selected from the group consisting of alkali hydroxide, alkali carbonate, ammonium or alkali salts of an organic acid, and a mixture of two or more thereof.

It may further be provided that the color indicator further comprises a carrier material, preferably an absorbent neutral carrier material. The carrier material may be open-pored or closed-pored. The individual components of the color indicator, such as the coloring substance or the reagent, may be contained in the pores.

In one embodiment, it may be provided that the color indicator is composed of a first solution of the color-forming substance in water or/and primary, secondary and/or tertiary alcohols and a second solution of a base, for example alkali hydroxide, in water or alcohols or mixtures thereof.

According to the invention, it is provided that the components contained in the color indicator react, alone or together with a change in the color of the color indicator, to the presence of substances contained in the material according to the invention.

In a further embodiment, it may be provided that the ampoule is a divisible ampoule comprising two or more parts that may be joined together to form the ampoule, wherein the color indicator is disposed on at least a portion of an inner wall of one of the parts that may be joined together to form the ampoule.

According to the invention, it can be provided that the ampoule can be closed in an airtight and/or air-tight manner.

For example, it can be provided that the ampoule consists of three different parts that are connected to each other in an airtight manner. The connection of the individual parts of the ampoule may be realized in various ways, such as by screwing the parts together. It may be provided that on an inner wall (or a part thereof) of a part (or parts) of the divisible ampoule the color indicator is arranged. Here, the inner wall is the part which is arranged towards the inside of the ampoule after the parts have been assembled and connected. Here it can be provided that the carrier is arranged on the inner wall of the ampoule or a part thereof, for example by an adhesive connection between the carrier and the inner wall. On the carrier, towards the interior of the ampoule, the remaining components of the color indicator, in particular the color-forming substance, are then arranged to be brought into contact with the material or components released from this material, for example with substances released from the hemp plant.

In one embodiment, it may be provided that the part on the inner wall of which the color indicator is at least partially arranged is a spacer disc which can be connected to one or more further parts of the divisible ampoule by screwing.

In one embodiment, it may be provided that the individual components of the divisible ampoule comprise disc-shaped screwable rings that can be connected to each other on both sides.

In a further embodiment, it may be provided that the ampoule, in particular the interior of the ampoule, is corrosion resistant, in particular to acids and bases.

In a further embodiment, the ampoule may be transparent. In particular, it can be provided that the ampoule is made of borosilicate glass. The transparency of the ampoule allows the color change of the color indicator to be easily evaluated optically and, if necessary, additionally stored accordingly and compared with data stored in a database as a reference.

In a further embodiment, it may be provided that the ampoule is evacuated.

The task is further solved by a method for qualitative and/or quantitative detection of one or more substance(s) contained in the hemp plant, comprising the steps of: a) providing a kit according to the invention; b) bringing the material or parts thereof into contact with the color indicator; and c) detecting a change in the color of the color indicator.

In this regard, it may be provided that bringing the material or portions thereof into contact with the color indicator comprises heating the material or portions thereof in the vial. In this regard, it may be provided that the heating comprises inductive heating.

In a further embodiment, it may be provided that detection is performed using a color comparison scale, an optical sensor, a chemical sensor, or two or more thereof.

Likewise, it may be provided that the method further comprises a step, after detection, comprising matching information obtained by the detection with data stored in a database.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

In the following, the invention will be described with reference to the drawings on the basis of specific embodiments. It is to be understood here that the reference to the specific embodiments serves only to illustrate the invention. The features of the specific embodiments are not necessarily limiting for the invention, but may, in particular in combination with the embodiments mentioned in the foregoing, contribute to the advantageous realization of the invention.

FIG. 1: schematic representation of an ampoule according to one embodiment of the invention;

FIG. 2: schematic representation of an ampoule according to a further embodiment of the invention; and

FIG. 3: schematic representation of an ampoule according to a further embodiment of the invention.

### DETAILED DESCRIPTION

Example embodiments will now be described more fully with reference to the accompanying drawings.

The invention relates to a method for quality testing of pharmaceutical cannabis, in which an ampoule containing a material, such as cannabis flowers or parts thereof, can be designed in such a way that the ampoule itself, or by combining it with additional elements, ensures the appropriate tests for marketing by the pharmacist, and test certificates can be verifiably documented and issued in accordance with pharmaceutical rules.

According to the method of the invention, it may be provided that the cannabis flowers or parts thereof are hermetically packaged immediately after harvesting and sealed in a special transparent ampoule, preferably made of borosilicate glass, in such a way that the active ingredient can only be removed by destroying the ampoule. Within the ampoule, a chemical analytical assay can be performed using various tools described herein. The results may be applied as data to appropriate bar codes or the like. Other corresponding relevant information is: e.g., cultivation, harvesting, processing, quality testing, storage, packaging, is of course also present in a tamper-proof manner. This can be achieved by burning a laser code into the surface. The data can be read out in a docking station.

With the new method, which is described in more detail in the claims, it is possible to perform quality tests even while the plant is growing. It is conceivable that the cannabis flower in the ampoule is examined during growth.

Chemical tests take place in or with the ampoule. The ampoule can be designed in such a way that the test elements with corresponding reagents form part of the ampoule or can be screwed onto the ampoule.

The kit described in the claims ensures compliance with the usual requirements, in particular:

Consistent product quality

Substances contained

Information on the concentration of the active substances

Avoidance of any kind of contamination

Traceability

The kit according to the invention and the method according to the invention enable skilled personnel, pharmacists, physicians or clinicians to chemically detect phenol-like substances, in particular cannabinoids and materials containing such substances.

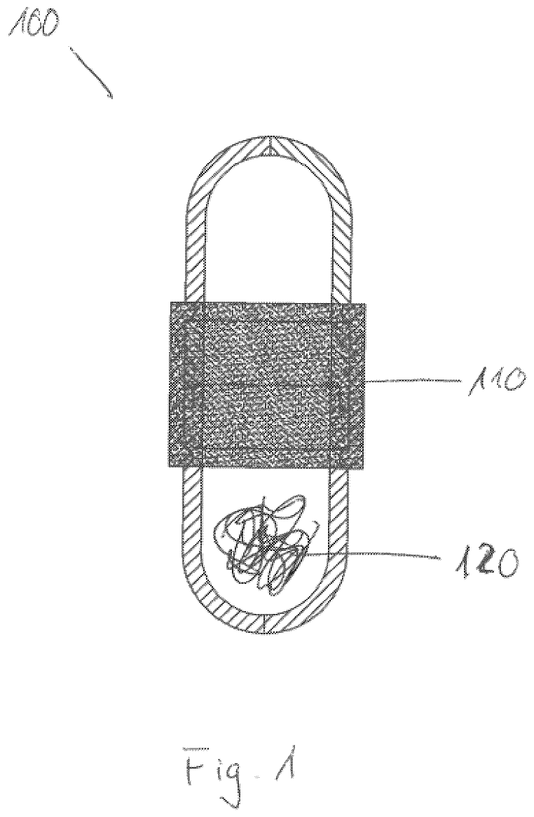

A color-forming reagent can be added directly to the sample, for example in pretreated or untreated form, even without prior drying. This can be achieved by a multi-part ampoule 100, as shown in FIG. 1, comprising an annular adapter 110 in a screw-on manner. The annular adapter 110 includes, in its ring-filling surface, the color indicator necessary for analyzing the material 120.

The color indicator can be used as a solution of a color-forming substance and the reagent with or without the addition of organic solvents.

The color-forming substance is preferably presented in the form of dilute solutions of the color-forming substance in water and/or primary, secondary and tertiary alcohols with or without the addition of organic solvents such as saturated and unsaturated hydrocarbons, halogenated hydrocarbons, ethers, ketones, carboxylic acid esters and/or aromatic hydrocarbons.

The reagent is preferably a base, such as an alkali hydroxide and/or an alkali carbonate, an ammonium or alkali salt of an organic acid or mixtures thereof, optionally substituted by one or more organic radicals, e.g. alkyl groups, and may be present in the form of a dilute solution together with the color-forming substance in water and/or primary, secondary and alcohols or mixtures thereof. Where appropriate, ammonium or alkali salts of organic acids such as acetic acid, propionic acid, butyric acid, malic acid, sorbic acid, fumaric acid, benzoic acid, phenylacetic acid, phthalic acid, naphthylacetic acid or mixtures thereof may be used.

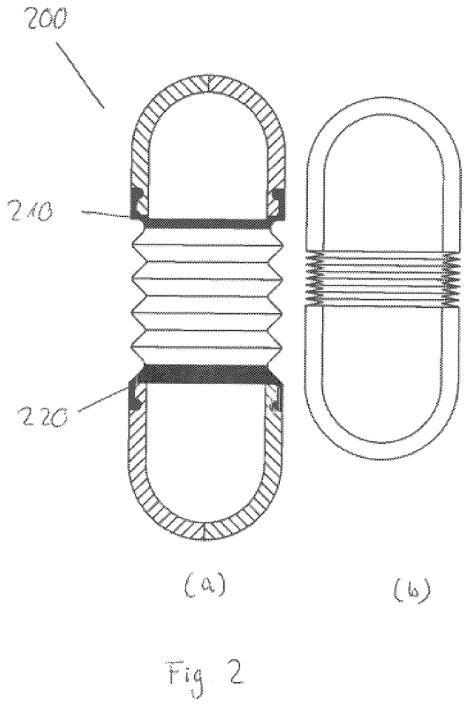

Surprisingly, it has been possible to develop a method in which specially selected color reactions are modified in such a way that the components relevant to the color reaction are added directly to the solvent acting as extraction agent and are used directly in the chemical detection reaction. In the ampoule 200 shown in FIG. 2, this is achieved by pressing the test material (not shown), in this case cannabis flowers, into the ampoule 200 between a first spacer ring 210 and a second spacer ring 220. In FIG. 2a, the ampoule is shown in a non-compressed state, whereas FIG. 2b shows the same ampoule in a compressed state.

The color indicator and its color change can be evaluated with appropriate optical and chemical sensors.

According to the aperture measurement method, it is achieved that the predominant phenol body can be made individually visible to the eye by developing a specific, clearly distinguishable color tone and can thus be directly detected in a previously unknown, simple and fast way. The fact that the procedure used takes place within the ampoule means that external influences are excluded. By refining the procedure with the aid of electronic optical sensors, it is possible to collect and store the data. Thus, an additional optimal, particularly easy to read gradation, as well as a permanent lasting storage of the resulting color tones is achieved. In this way, for the first time, it is also possible to subject fresh, untreated sample material and even fresh plants in pre-treated or untreated form directly to the examination and to analyze them for phenolic or cannabinoid components, even without prior drying, e.g. in the field. A comparison of the recorded data in the course of growth is thus possible. The method according to the invention has proved particularly useful in the detection of cannabinoids. This makes it possible for the first time to distinguish between drug and industrial hemp in young or adult, male or female plants from an age of two weeks. Furthermore, the determination of the degree of maturity of hemp plants can be carried out in a fast and simple way even directly on fresh plants. The fields of application of the method according to the invention include cannabis research and medicine, cannabis consumption (quality control) and regulatory requirements for the pharmacist, physician, etc. Furthermore, the method can be used to detect cannabis preparations in biological sample materials. By special modifications of the reagents and the procedures, new methods for selective detection of single specific cannabinoids could be found.

Due to the simple design of the method and the reagents required for it, this method is particularly suitable for analysis packs that can be used directly on site. By means of software-supported, photometric measurement of the absorption main maxima of the developed color shades, the methods can also be used for quantitative determination of the detected phenolic bodies. Data storage and comparison possibilities extend the safety in the handling of medical cannabis.

In the reagent solution, monohydric or polyhydric alcohols alone or as a mixture are preferably used as solvents, which on the one hand guarantee optimum extraction of the active ingredients and on the other hand also good dissolving properties for the components of the dressing board used, which are responsible for the compound, and a trouble-free color reaction. The components of the color dictator necessary for color development were designed in such a way that they only have to be added to the reagent mixture in drop quantities for the purpose of the simplest and most effective handling. By adding salts of organic acids to the reagent solution, a better readability of the developed color shades as well as an improved shelf life of the same can be achieved.

With the appropriate storage of the color shades, a database can be created that allows the assignment of the hemp plants according to location and origin.

The invention will be explained and illustrated with reference to the following examples, but without being limited to them.

Examples of color representations:
Hemp fresh plants immature drug hemp purple reddish mature blue green
Mature EU industrial hemp purple reddish
Thymol deep blue
Cannabidiol (CBD) purple pink
Tetrahydrocannabiol green blue
Cannabinol (CBN) blue

For the determination of the degree of maturity, fresh, not dried plant material is used, since drying can change the cannabinoid content (e.g. conversion of cannabidiol to THC). To distinguish industrial or drug hemp, fully formed fingering normal leaves (fresh or dried) of the plant are used rather than the inflorescences or fruiting units. By testing the normal, fingered leaves of a given young plant, at least two weeks old, its maximum achievable future cannabinoid spectrum can be determined. If the developed hues are too intense and thus difficult to read, the test should be repeated with a reduced sample size. In turn, too small sample amounts can result in pale, indistinct hues that shift into the yellow. In this case, the test should be repeated with a larger sample quantity.

### Performance of the Tests

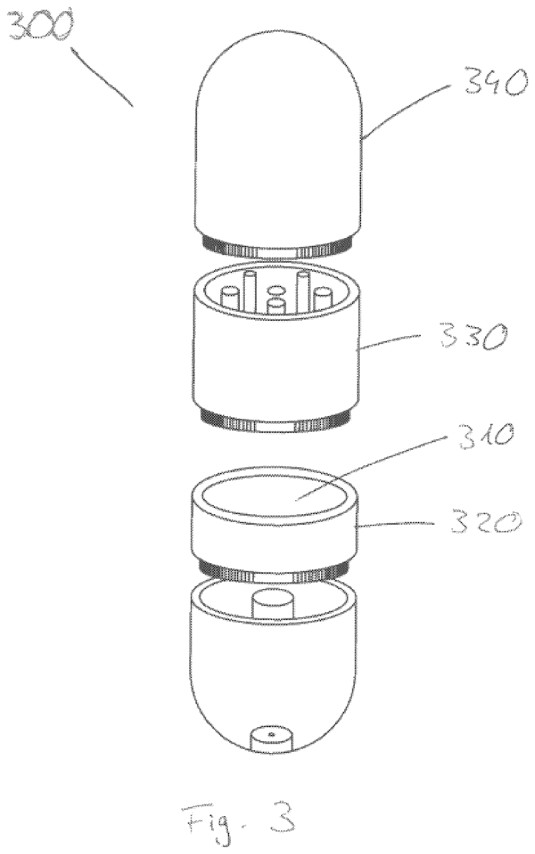

A small amount of the samples is placed on a ring-shaped reagent-soaked surface 310 of a portion of the divisible ampoule 300 shown in FIG. 3. A spacer 320 containing a solution of the colorant is sealed with a spacer 330 containing a solution of the reagent with a plastic cap 340 and shaken several times within about 1 min. Within a minute, the resulting coloration of the supernatant solution can be read. The best results are visible in translucent daylight or against a bright surface. A very accurate result is of course obtained by software-controlled evaluation with the aid of a photospectrometric device.

The features disclosed in the foregoing description and the appended claims may, separately or in combination, be subject matter for realizing aspects of the disclosure made in the independent claims in various forms thereof.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for qualitative and/or quantitative detection of one or more substances contained in a hemp plant, comprising:
(a) Providing a kit, the kit including:
an ampoule, the ampoule being transparent;
a material including at least part of a hemp plant;
a color indicator configured to react by contacting the material and
change the color of the color indicator;
(b) transporting the ampoule to a third party;
(c) heating the ampoule;
(d) mixing the material and the color indicator together by the third party; and
(e) detecting a change in the color of the color indicator by the third party.

2. The method of claim 1, wherein the providing comprises arranging the material and the color indicator separately and unmixed in the ampoule.

3. The method of claim 1, the ampoule being divisible and having a plurality of parts, wherein the providing comprises separately arranging the material in one part of the ampoule and the color indicator in the other part of the ampoule.

4. The method of claim 3, wherein the color indicator is a color-forming substance and a reagent, wherein the providing comprises separately arranging the material in a first part of the ampoule, the color-forming substance in a second part of the ampoule and the reagent in a third part of the ampoule.

5. The method of claim 1, wherein the providing comprises sealing the ampoule containing the material and the color indicator.

6. The method of claim 5, wherein the sealing comprises evacuating the ampoule containing the material and the color indicator.

7. The method of claim 1, wherein the heating comprises inductive heating.

8. The method of claim 1, wherein the detecting is performed using a color comparison scale, an optical sensor, a chemical sensor, or two or more thereof.

9. The method of claim 8, wherein the detecting is performed through the transparent ampoule.

10. The method of claim 1, further comprising, after step (e), matching information obtained by the detection in step (e) with data stored in a database.

11. The method of claim 1, further comprising after step (e), creating a database by storage of detected color shades of the material.

12. The method of claim 11, wherein the color shades are stored in the database together with a location or origin of the material.

13. The method of claim 1, further comprising, after step (e), applying the detected results as data to appropriate bar codes or the like.

14. The method of claim 13, wherein the data comprise information corresponding to the detected results about at least one of the following: cultivation, harvesting, processing, quality testing, storage, packaging.

15. The method of claim 13, wherein the applying comprises burning a laser code into a surface of the ampoule.

16. The method of claim 13, further comprising, after applying the detected results, reading out the applied data in a docking station.

17. The method of claim 1, wherein the ampoule is at least partially made of borosilicate glass.

18. The method of claim 1, wherein the material comprises a cannabis flower, marijuana, hashish, hashish oil, at least one cannabinoid, or a mixture thereof.

19. The method of claim 1, wherein the material is provided in fresh or dried form.

20. The method of claim 1, wherein the color indicator comprises a color-forming substance preferably selected from the group consisting of true black, true blue salt, dibromoquinone chlorimide, dichloroquinone chlorimide, vanillin, salicylaldehyde, formaldehyde, acetaldehyde, p-dimethylaminobenzaldehyde, diethylaminobenzaldehyde, ferric chloride, aminophenol, potassium ferricyanide, and a mixture of two or more thereof.

21. The method of claim 1, wherein the color indicator comprises at least one solvent preferably selected from the group consisting of water and monohydric or polyhydric alcohols.

22. The method of claim 20, wherein the color indicator further comprises a reagent that assists in reacting the material with the color-forming substance, wherein the reagent is preferably a basic compound, more preferably selected from the group consisting of alkali hydroxide, alkali carbonate, ammonium or alkali salts of an organic acid, and a mixture of two or more thereof.

23. The method according to claim 20, wherein the color indicator further comprises a carrier material, preferably an absorbent neutral carrier material.

24. The method of claim 1, wherein the providing comprises disposing the color indicator on at least a portion of an inner wall of the ampoule.
